(19) United States
(12) Patent Application Publication
(10) Pub. No.: US 2005/0256427 A1
(43) Pub. Date: Nov. 17, 2005

(54) Precision contoured exo/endocervical cell sampler

(75) Inventor: Yoon Ngen Moo, Kuala Lumpur (MY)

Correspondence Address: VOLPE AND KOENIG, P.C., UNITED PLAZA, SUITE 1600, 30 SOUTH 17TH STREET, PHILADELPHIA, PA, 19103, US

(73) Assignee: Modern Medical Equipment Mfg., Kwai Chung, NT (HK)

(21) Appl. No.: 10/519,019

(22) PCT Filed: Jun. 21, 2003
(86) PCT No.: PCT/SG03/00153

§ 371 Date: Dec. 21, 2004

(30) Foreign Application Priority Data
Jun. 21, 2002 (MY) PI20022342

(51) Int. Cl. A61B 10/00

(52) U.S. Cl. 600/570

(57) ABSTRACT

An L-shaped, precision contoured EXO/ENDOcervical cell sampler for collecting representative cells is invented for pre-malignant and malignant diagnosis. The said cell sampler comprises of an elongated and rounded stick-shaped stem with a slightly thickened handle means at one end, and a flattened connecting means havig two protrusions at the other end. An L-shaped configuration at the root of said connecting means has one vertical ENDOcervical contact portion and one horizontal EXOcervical contact portion, which directly hug to the transformation zone thereby allowing a through and complete 360 DEG rotatinal sweeping-up of all the truly representatives cells. When said cell sampler is withdrawn from the cervix, the vertical ENDOcervical contact portion is subsequently bent and both the EXO- and ENDOcervical portions are swept across a microscope slide for cytological microscopic examination.

### FIELD OF INVENTION

The invention relates to a specific device for collecting representative cell samples from exocervix and endocervix for cytological microscopic examination, particularly for the purpose of pre-malignant and malignant diagnosis.

### BACKGROUND OF THE INVENTION

Occurrence of uterine cervical cancer is high among women and the rate has been increasing. The disease becomes the main cause of cancer death among women in Malaysia. Of vital important, women are advised to have periodical medical pelvic examinations for early detection and prevention of uterine cervical cancer.

Many devices have been invented for sampling of representative cells from the uterine endocervix and exocervix for the purpose of pre-malignant and malignant diagnosis. Such devices are generally having an elongated stem with a portion at one end, which is designed as a swab, spatula or a brush. However, drawbacks have been reported from these conventional techniques. The swab technique has the disadvantages that the stick may break when abrasive force is applied to enable specimen sampling and extra time-consuming step is required to examine the mixing of desired and undesired cells on a microscope slide. In addition the cotton buds swab stick is also unsatisfactory in retaining and transferring of cellular samples due to its rather tightly-wound and very absorbent ball-like surface. It has also a very limited contact with the exocervix.

The spatula technique enables cell sampling even from deeper cell layers, however, such device may cause post-exam bleeding or abrasion at the sampling spot. In addition the wooden or plastic spatulas are too stiff and rigid to provide an even and thorough scraping of the unevenly contoured uterine cervix. It is also less efficient in transferring the cellular samples onto the glass slides due to its inflexible L-shaped end portion.

Cellular samples obtained by the brush technique are usually overly blood-stained and a fair number of critical cervical cells are being trapped in between the bristles and hence do not get transferred fully onto the glass slides and therefore are wasted and lost for diagnosis. The brush device has also a very limited contact with the exocervix.

### SUMMARY OF THE INVENTION

The primary object of this invention is to provide a new and improved cell sampler to collect representative cell samples from the exocervix and endocervix for cytological microscopic examination. It involves only a simple “one-step” operation where a single insertion into the cervical canal can obtain both exocervical and endocervical cell samples.

Another object of this invention is to provide a cell sampler that holds securely at the exocervix and prevent excessive penetration into the internal os.

Still another object of this invention is to provide a cell sampler having an endocervical contact portion that is safe in use, thereby provides more comfort and less abrasive when making a complete 360 DEG rotational sweeping-up of all the representative cells.

Yet another object of this invention is to provide a cell sampler that is capable of collecting cells from deep inside body cavities due to the semi rigid vertical reach of the endocervical contact portion.

A further object of this invention is to provide a cell sampler that allows combined yet separated cell samples from both exocervical and endocervical regions with just a single swipe onto a microscope slide where these cellular portions are clearly visible.

A still further object of this invention is to provide a cell sampler having a slightly thickened-handle to enhance better finger grip and optimal rotational manipulation.

These and further objects, features and advantages of the present invention will become apparent from the following description when taken in connection with the accompanying drawings which, for purposes of illustration only, show the preferred embodiment in accordance with the present invention.

### BRIEF DESCRIPTION OF THE DRAWINGS

FIG. 1 is a perspective view of the present cell sampler for sampling purpose.

FIG. 2 is a perspective view of the present cell sampler for spreading purpose.

FIG. 3 illustrates the transfer of samples onto a microscope slide.

FIG. 4 illustrates the rotational mechanism of the vertical contact portion of the present cell sampler.

### DETAILED DESCRIPTION OF THE INVENTION

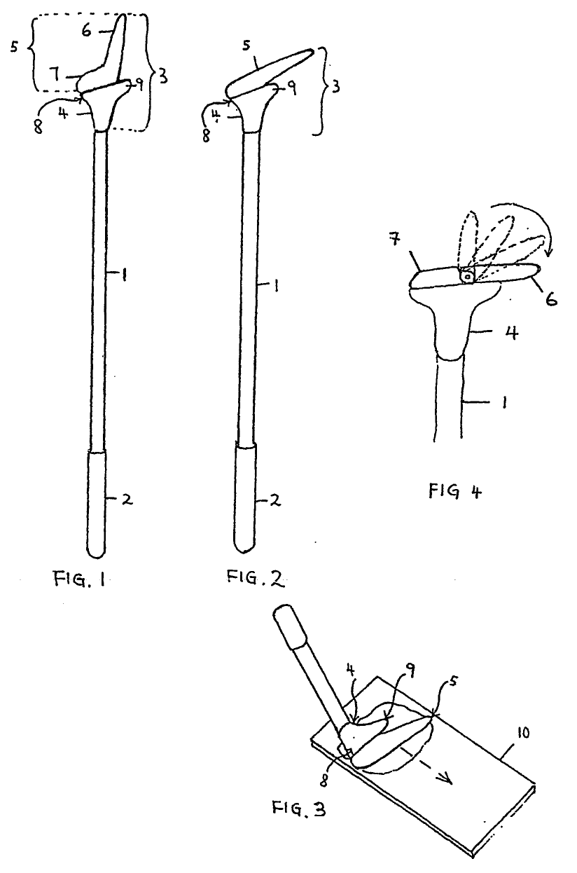

With reference to the FIG. 1, the drawing shows in embodiment comprises an elongated and rounded stick-shaped stem (1) having a slightly thickened handle means (2) at bottom end to enhance better finger grip and optimal rotational manipulation. At the top end of said stem (1) is connected to an abrading means (3). The said abrading means (3) comprises a connecting means (4) and a functional L-shaped cell collecting means (5). The functional L-shaped cell collecting means (5) comprising of a vertical ENDOcervical contact portion (6) therein to aid in insertion of said cell sampler into the endocervix and configured to scrape cytology cell samples onto its surface when said cell sampler is rotated; and a horizontal EXOcervical contact portion (7) therein to aid in hugging the exocervix and scraping cytology cell samples onto its surface when said cell sampler is rotated.

The said vertical endocervical contact portion (6) is connected to the horizontal exocervical contact portion (7) by attachment means, preferably a predetermined, semi-tightened ball joint thus allowing the said vertical contact portion to has a 90 degree free moving angle with the respect to the horizontal exocervical contact portion (7) as shown in FIG. 4. The vertical endocervical contact portion (6) can stay critically upright while performing its cell sampling rotational scraping function and also enable to made instantly collapsible to a horizontal position onto microscope slide with just very slight finger pressure thus accomplishing its excellently designed objective of spreading the exo/endocervical cells speedily, smoothly and easily onto a microscope slide without the slightest hindrances. The attachment means also could be any fasteners, screws, snaps, clamps, clips, nuts or other such equivalents that could used to secure one surface to another and rotational movements with respect to the horizontal exocervical contact portion (7) are allowed.

The connecting means (4) having one end which is connected to said stem (1) is rounded in cross sectional shape and the other end is a flattened end. Supported one top of the said flattened end is said functional L-shaped cell collecting means (5). The said flattened end of the connecting means (4) comprising of a supporting protrusion (8) at one edge of said flattened end and a retaining protrusion (9) at the other edge of said flattened end. The said EXOcervical contact portion (7) is attached to the supporting protrusion (8) of said connecting means (4).

The functional L-shaped cell collecting means (5) is coated with resilient material such as sponge, foam, fibre, silicon, PVC film, rubber, soft plastics and the like, thereby provide more comfort and less abrasive, yet direct and gently hugging to the critical transformation zone (T-zone), thereby allowing a thorough and complete 360 DEG rotational sweeping-up of all the truly representative cells. The retaining protrusion (9) of said connecting means (4) being configured to prevent excessive penetration of said cell sampler into the endocervix and lend resistance to the collapsible of said vertical ENDOcervical contact portion (6).

FIG. 2 shows a flattened portion of EXOcervical and ENDOcervical contact portions. After a smear sample has been taken from the cervical canal, the said cell sampler is withdrawn from the cervix and the vertical ENDOcervical contact portion (6) will immediately be bent and collapsed on top of a microscope slide (10) as shown in FIG. 3, becoming a horizontal, straight and flat spreading strip to be gently swept across the full length of the microscope slide (10), demonstrating clearly a combined yet separated cellular portions from both the exocervical and endocervical regions.

The invention is advantageously for use in a simple “one-step” operation involving only a single insertion for sampling cells from exocervix and endocervix and obtain representative cells from both regions to be included on the same microscope slide.

It is to be understood that the present invention may be embodied in other specific forms and is not limited to the sole embodiment described above. However modification and equivalents of the disclosed concepts such as those which readily occur to one skilled in the art are intended to be included within the scope of the claims which are appended thereto.

1) A sampler for sampling cervical cells comprising of;
an elongate handle;
first collecting means attached to one end of said handle for hugging the exocervix and scraping cytology cell samples;
second collecting means attached to first collecting means for collecting cytology cell samples from the endocervix area;
wherein said first and second collecting means are pivotally connected.

2) A sampler as claimed in claim 1 wherein the first and second collecting means each have a resilient surface to collect the cell samples.

3) A sampler as claimed in claim 1 wherein the first and second collecting means each have a bristle surface to trap the cell samples.

4) A sampler as claimed in claims 2 and 3 wherein the surfaces are made of polymeric material.

5) A sampler as claimed in claim 2 and 3 wherein the resilient surface is made of sponge, foam or fibre.

6) A sampler as claimed as claim 1 wherein when in collecting position the first collecting means is substantially right angle with the second collecting means.

7) A sampler as claimed in claim 1 wherein when in smearing position the surfaces of the first and second collecting means are in the same plane to enable a wider spread of the collected cell samples onto microscope slide.

8) A sampler as claimed in claim 7 wherein in smearing position the end of handle adjacent the second collecting means has a slight extension to prevent the said second collecting means from pivoting beyond the same planar as the first collecting means.

9) A cell sampler which includes a handle, first collecting means attached to one end of said handle for hugging the exocervix and scraping cytology cell samples, second collecting means pivotly attached to first collecting means for collecting cytology cell samples from the endocervix area;
characterised in that;
said first and second collecting means are pivotally connected so as to be movable relatively to one another between smearing position in which they are generally L-shaped configuration and a smearing position in which they are in a straight line.
